(19) United States
(12) Patent Application Publication
(10) Pub. No.: US 2026/0166329 A1
(43) Pub. Date: Jun. 18, 2026

(54) VECTOR POTENTIAL GENERATION DEVICE, VECTOR POTENTIAL COIL ARRANGEMENT METHOD, VECTOR POTENTIAL TRANSFORMER, AND CONTACTLESS POWER SUPPLY SYSTEM

(71) Applicants: Sumida Corporation, Tokyo (JP); National University Corporation, Iwate University, Iwate (JP)

(72) Inventors: Masahiro DAIBO, Morioka (JP); Asahi Sato, Morioka (JP); Tsuanki Kaneko, Miyagi (JP); Masaki Saito, Miyagi (JP); Thitaporn Detmod, Miyagi (JP); Kenji Terao, Miyagi (JP); Yoshiharu Yoshii, Miyagi (JP)

(21) Appl. No.: 18/849,870

(22) PCT Filed: Apr. 10, 2023
(86) PCT No.: PCT/JP2023/014505

§ 371 Date: Sep. 23, 2024

(30) Foreign Application Priority Data
Jun. 23, 2022 (JP) 2022-100853

(51) Int. Cl. A61N 2/02; H01F 5/00; H01F 7/20

(52) U.S. Cl. CPC A61N 2/02; H01F 5/00; H01F 7/20

(57) ABSTRACT

A vector potential coil is a vector potential which is a solenoid coil extending along a curved coil axis. A ferromagnetic member extends along the coil axis within the solenoid coil. A power supply device conducts a current through the vector potential coil. Further, the vector potential coil and the ferromagnetic member have an opening in a circumferential direction.

### CROSS REFERENCE TO RELATED APPLICATIONS

This application is a 371 U.S. National Phase of International Application No. PCT/JP2023/014505, filed on Apr. 10, 2023, which claims priority to Japanese Patent Application No. 2022-100853, filed Jun. 23, 2022. The entire disclosures o f the above applications are incorporated herein by reference.

### BACKGROUND

### Technical Field

The present invention relates to a vector potential generation device, a vector potential coil arrangement method, a vector potential transformer, and a contactless power supply system.

### Related Art

In recent years, a vector potential generation device has been developed (for instance, refer to Patent Documents 1 and 2). The vector potential generation device generates a vector potential by conducting a current through a vector potential coil that is formed by spiraling (circulating) a solenoid coil. further, a vector potential transformer that transmits power by using a vector potential and stimulation to a deep part of a living body by using a vector potential have also been proposed.

Further, a vector potential generation device has also been developed (for instance, refer to Patent Document 2). The vector potential generation device makes an internal region of a vector potential coil (a hollow region that is formed by windings of a solenoid coil, and in other words, by winding around a coil axis) to be a state of substantially absence of a magnetic field (a substantially magnetic field-free state) by using a return current conductor being connected to the vector potential coil in series.

Furthermore, a vector potential detection device has also been developed (for instance, refer to Patent Document 3). The vector potential detection device detects a vector potential by utilizing a state in which a voltage is induced by a temporal change (varying in time) of the vector potential.

### PRIOR ART DOCUMENTS

### Patent Documents

Patent Document 1: International Patent Publication Number WO2015/099147.
Patent Document 2: The specification of Japanese Patent Number 6205572.
Patent Document 3: The specification of Japanese Patent Number 6950925.

However, since the above-mentioned vector potential coil circulates (goes around) one or more rounds, an application target to which a generated vector potential is applied with a good intensity (for instance, a secondary-side conductor) is required to be arranged in an internal region of the vector potential coil that is in an approximately annular shape. Thus, the application target, to which the generated vector potential is applied, is limited to those that can be inserted into the internal region of the vector potential coil.

The present invention has been made in consideration of the above problems. The present invention has an object that is to obtain a vector potential generation device and a vector potential coil arrangement method that have fewer restrictions on an application target to which a vector potential is applied, and a vector potential transformer and a contactless power supply system that can utilize the vector potential generation device.

### SUMMARY

A vector potential generation device according to the present invention includes a vector potential coil which is a solenoid coil extending along a curved coil axis, a ferromagnetic member extending along the coil axis within the solenoid coil, and a power supply device that conducts a current through the vector potential coil. The vector potential coil and the ferromagnetic member have an opening in a circumferential direction.

A vector potential coil arrangement method according to the present invention includes arranging a vector potential coil and a ferromagnetic member at a position at which a vector potential is generated in a brain of a human body via a support body (a support) without attaching (adhering, sticking, or affixing) the support body to a head of the human body. The support body supports the vector potential coil, which is a solenoid coil extending along a curved coil axis, and the ferromagnetic member extending along the coil axis within the solenoid coil.

A vector potential generation device according to the present invention includes a plurality of vector potential coils, which are a plurality of solenoid coils extending along their respective coil axes, and a power supply device that conducts a current to the plurality of vector potential coils. Further, the plurality of vector potential coils are arranged along a linear arrangement direction or a curved arrangement direction.

A vector potential coil arrangement method according to the present invention includes arranging a plurality of vector potential coils at a position at which a vector potential is generated in a brain of a human body via a support body (a support) without attaching (adhering, sticking, or affixing) the support body to a head of the human body. The support body supports the plurality of vector potential coils which are a plurality of solenoid coils extending along coil axes, respectively.

A contactless power supply system according to the present invention includes any of the above-mentioned vector potential generation devices and a power receiving side device. Further, the power receiving side device senses the vector potential generated by the vector potential generation device and has a secondary conductor member, in which a secondary voltage is induced by the vector potential, and a power supply circuit that supplies power obtained from the secondary voltage to a load.

A vector potential transformer according to the present invention includes a vector potential coil, which is a solenoid coil extending along a curved coil axis, a ferromagnetic member extending along the coil axis within the solenoid coil, and a secondary conductor member that senses the vector potential and in which a secondary voltage is induced by the vector potential. The vector potential coil and the ferromagnetic member have the opening in a circumferential direction.

A vector potential transformer according to the present invention includes a plurality of vector potential coils, which are a plurality of solenoid coils extending along coil axes, respectively, and a secondary conductor member that senses the vector potential generated by the plurality of vector potential coils and in which a secondary voltage is induced by the vector potential. Further, the plurality of vector potential coils are arranged along a linear arrangement direction or a curved arrangement direction.

### Effects of the Invention

According to the present invention, it is possible to obtain a vector potential generation device and a vector potential coil arrangement method that have fewer restrictions on an application target to which a vector potential is applied, and a vector potential transformer and a contactless power supply system that can utilize the vector potential generation device.

### BRIEF DESCRIPTION OF THE DRAWINGS

FIG. 1 is a block diagram that shows a configuration of a vector potential generation device 10 according to an embodiment of the present invention.

FIG. 2 is a diagram that shows an example of a vector potential coil with respect to a vector potential coil device 1 shown in FIG. 1.

FIG. 3 is a diagram that shows an example of a vector potential coil device 1 according to a first embodiment.

FIG. 4 is a diagram that shows an example of an application of a vector potential by a vector potential generation device 10 according to the first embodiment.

FIG. 5 is a block diagram that shows a configuration of a contactless power supply system according to a second embodiment of the present invention.

FIG. 6 is a diagram that shows an example of a vector potential coil device 1 and a secondary conductor member 21 with respect to the contactless power supply system shown in FIG. 5.

FIG. 7 is a diagram that shows a configuration of a vector potential coil device 1 with respect to a vector potential generation device 10 according to a fourth embodiment of the present invention.

FIG. 8 is a diagram that shows a configuration of a vector potential coil device 1 with respect to a vector potential generation device 10 according to a fifth embodiment of the present invention.

FIG. 9 is a diagram that shows a configuration of a vector potential coil device 1 with respect to a vector potential generation device 10 according to a sixth embodiment of the present invention.

FIG. 10 is a diagram that shows a configuration of a vector potential coil device 1 with respect to a vector potential generation device 10 according to a seventh embodiment of the present invention.

FIG. 11 is a diagram that shows an example of a bed that incorporates the vector potential generation device 10 according to the seventh embodiment.

FIG. 12 is a diagram that shows a configuration of a vector potential coil device 1 with respect to a vector potential generation device 10 according to an eighth embodiment of the present invention.

FIG. 13 is a diagram that shows a configuration of a vector potential coil device 1 with respect to a vector potential generation device 10 according to a ninth embodiment of the present invention.

FIG. 14 is a front view that shows an example of a vector potential coil with respect to a vector potential coil device 1 according to a tenth embodiment of the present invention.

FIG. 15 is a top view that shows an example of the vector potential coil with respect to the vector potential coil device 1 according to the tenth embodiment of the present invention.

FIG. 16 is a side view that shows an example of the vector potential coil with respect to the vector potential coil device 1 according to the tenth embodiment of the present invention.

### DETAILED DESCRIPTION

Embodiments of the present invention will be explained below with reference to the drawings.

### First Embodiment

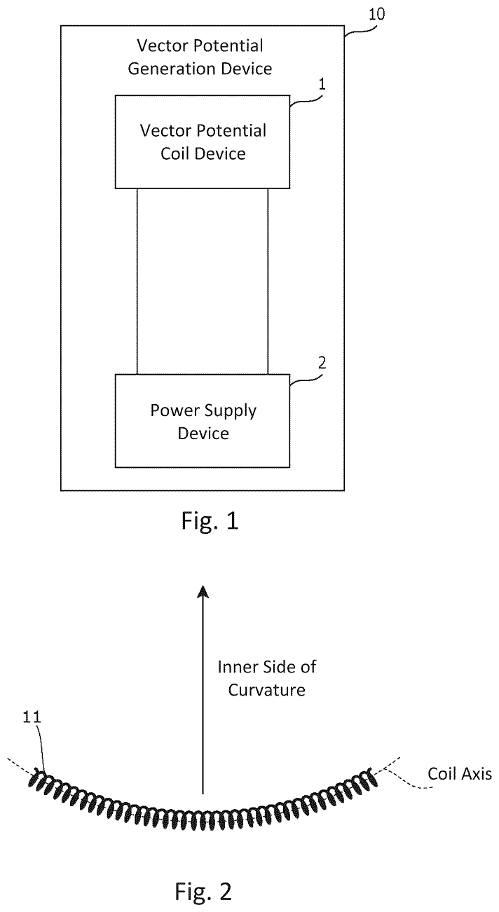

FIG. 1 is a block diagram that shows a configuration of a vector potential generation device 10 according to an embodiment of the present invention. The vector potential generation device 10 shown in FIG. 1 has a vector potential coil device 1 and a power supply device 2.

FIG. 2 is a diagram that shows an example of a vector potential coil with respect to the vector potential coil device 1 shown in FIG. 1. The vector potential coil device 1 has, as shown in, for instance, FIG. 2, the vector potential coil (also referred to as “a VP coil” below) 11. The VP coil 11 is a solenoid coil extending along a curved coil axis.

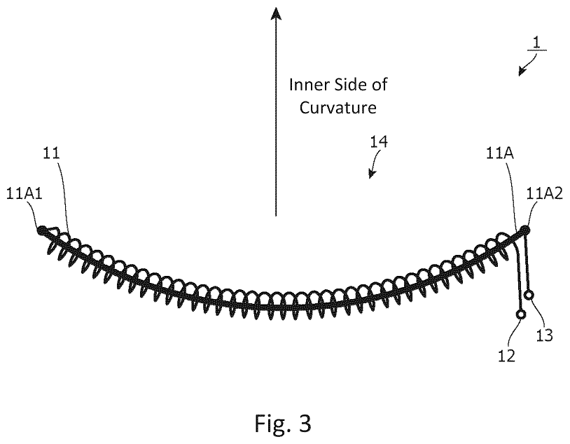

FIG. 3 is a diagram that shows an example of a vector potential coil device 1 according to a first embodiment. As shown in, for instance, FIG. 3, the vector potential coil device 1 has a ferromagnetic member 11A in addition to the above-mentioned VP coil 11. The ferromagnetic member 11A is in a shape that extends along the above-mentioned coil axis within the above-mentioned solenoid coil and is formed with a ferromagnetic material.

A vector potential due to a current that is conducted through the VP coil 11 is weakened as it moves away from the current. However, since the VP coil 11 and ferromagnetic member 11A are curved as mentioned above, an intensity of the vector potential becomes greater at an inner side (an inner direction) of the curvature (a curvature center in the case of a circular arc shape). Specifically, the vector potentials being generated by the current at each position of the VP coil 11 overlap at the inner side of the curvature. Further, since the vector potential is enhanced according to an effective magnetic permeability of the ferromagnetic member 11, the intensity of the vector potential becomes greater at the inner side of the curvature (a curvature center in the case of a circular arc shape).

The power supply device 2 shown in FIG. 1 generates a current based on power from, for instance, a commercial power supply or a battery (a primary battery or a secondary battery), and conducts that current (here, an alternating current (AC current) 4 a predetermined frequency) through the VP coil 11. Note that in a case in which the power supply device 2 conducts a current through the VP coil 11 by using the power from the battery, the vector potential generation device 10 may be a portable device that incorporates the battery.

In addition, as shown in, for instance, FIG. 3, the VP coil 11 and the ferromagnetic member 11A have an opening 14 in a circumferential direction. In other words, a coil axis of the VP coil 11 does not go around once (one revolution) or more.

For instance, the above-mentioned coil axis is in a circular arc shape. Further, an angle (central angle) from one end to the other end of the VP coil 11 (the coil axis of the VP coil) when viewed from a center of a circle including the coil axis (i.e., the circular arc) is less than 360 degrees. Similarly, an angle (central angle) from one end to the other end of the ferromagnetic member 11A when viewed from the center of the circle including the coil axis is less than 360 degrees. As a result, the opening 14 is formed. For instance, the central angle may be 180 degrees or may be less than 180 degrees. However, the larger the central angle is, the greater the intensity of the vector potential in the inner side of the curvature becomes. Thus, it is preferred that the central angle is large. The central angle is any angle greater than 0 degrees and less than 360 degrees, and may further be (a) any angle greater than 0 degrees and equal to or less than 180 degrees, (b) any angle greater than 0 degrees and equal to or less than 90 degrees, (c) any angle greater than 0 degrees and equal to or less than 45 degrees, or (d) any angle equal to or greater than 0.5 degrees and less than 360 degrees, and further, (e) any angle equal to or greater than 0.5 degrees and equal to or less than 180 degrees, (f) any angle equal to or greater than 0.5 degrees and equal to or less than 90 degrees, (e) any angle equal to or greater than 0.5 degrees and equal to or less than 45 degrees, (f) any angle equal to or greater than 0.5 degrees and equal to or less than 25 degrees, or (g) any angle equal to or greater than 2 degrees and less than 360 degrees, further, (h) any angle equal to or greater than 2 degrees and equal to or less than 180 degrees, (i) any angle equal to or greater than 2 degrees and equal to or less than 90 degrees, (j) any angle equal to or greater than 2 degrees and equal to or less than 45 degrees, (k) any angle equal to or greater than 2 degrees and equal to or less than 25 degrees, or (l) any angle equal to or greater than 5 degrees and less than 360 degrees, further, (m) any angle equal to or greater than 5 degrees and equal to or less than 180 degrees, (n) any angle equal to or greater than 5 degrees and equal to or less than 90 degrees, (o) any angle equal to or greater than 5 degrees and equal to or less than 45 degrees, or (p) any angle equal to or greater than 5 degrees and equal to or less than 25 degrees. Furthermore, when it is considered about attachment and detachment of an application target, to which the vector potential is applied, to and from the VP coil 11 from the inner side of the curvature, it is preferred that the opening 14 is to be large (in other words, a curvature radius of the above-mentioned coil axis and/or the above-mentioned central angle are determined according to the shape and size of the application target).

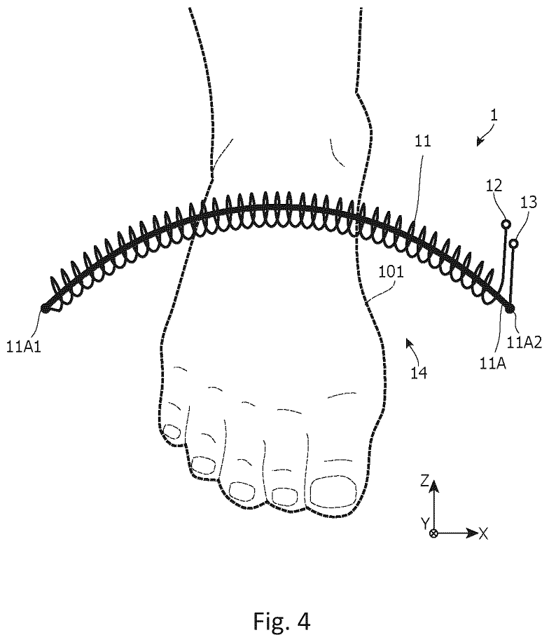

FIG. 4 is a diagram that shows an example of an application of the vector potential by the vector potential generation device 10 according to the first embodiment. As shown in, for instance, FIG. 4, the application target (in FIG. 4, a foot 101 of a human body), to which the vector potential is applied, is arranged in the opening 14.

In addition, in the first embodiment, the ferromagnetic member 11A is formed with a conductive material such as permalloy. Further, since one end of the VP coil 11 and one end (an end 11A1) of the ferromagnetic member 11A are electrically connected to each other, the ferromagnetic member 11A forms a path for a current. In addition, the power supply device 2 conducts a current through the VP coil 11 by applying a voltage to the other end of the VP coil 11 and the other end of the ferromagnetic member 11A. Here, the power supply device 2 conducts the current through the VP coil 11 applying the voltage to a terminal 12 being electrically connected to the other end of the VP coil 11 and a terminal 13 being electrically connected to the other end (an end 11A2) of the ferromagnetic member 11A.

In addition, since the coil axis of the VP coil 11 does not go around once (one revolution) or more, the distance between both ends of the VP coil 11 is large.

However, since the ferromagnetic member 11A is used as the current path and two of the terminals 12 and 13 are arranged on either end side of the VP coil 11, the area being encircled by the path through the wiring from the power supply device 2 to the VP coil 11 and the ferromagnetic member 11A is relatively narrow. As a result, an unnecessary magnetic field being generated due to the current flowing through the wiring can be suppressed.

Next, an operation of the vector potential generation device 10 according to the first embodiment will be explained.

The power supply device 2 applies a predetermined voltage to the above-mentioned terminals 12 and 13 of the vector potential generation device 10 so as to conduct a current through the VP coil 11 and the ferromagnetic member 11A.

A magnetic field is generated along the coil axis by the current being conducted through the VP coil 11. A vector potential is generated in parallel to the current. Further, an intensity of the vector potential in the inner side of the curvature of the VP coil 11 (i.e., in a periphery of the opening 14) becomes greater than the vector potential in an outer side of the curvature of the VP coil 11.

Therefore, the vector potential is effectively applied to the application target of the vector potential that is arranged in the opening 14.

As mentioned above, according to the above-mentioned first embodiment, the VP coil 11 is the solenoid coil extending along the curved coil axis. The ferromagnetic member 11A extends along the coil axis within the solenoid coil. The power supply device 2 conducts the current through the VP coil 11. In addition, the VP coil 11 and the ferromagnetic member 11A have the opening 14 in the circumferential direction (that is, in the circumferential direction of the VP coil 11 and the ferromagnetic member 11A that go around less than once (one revolution)).

As a result, because it is possible to arrange the application target of the vector potential at the opening 14 or in an internal region of the VP coil 11 via the opening 14, there are fewer restrictions on the application target of the vector potential.

For instance, in the case in which a vector potential is applied to such as a shoulder of a human body, using a VP coil being in an annular shape requires a relatively large VP coil with a hollow portion in which a torso of the human body goes. However, by using the VP coil 11 as shown in the first embodiment, it is possible to effectively apply a vector potential to such a location with a relatively small VP coil 11.

### Second Embodiment

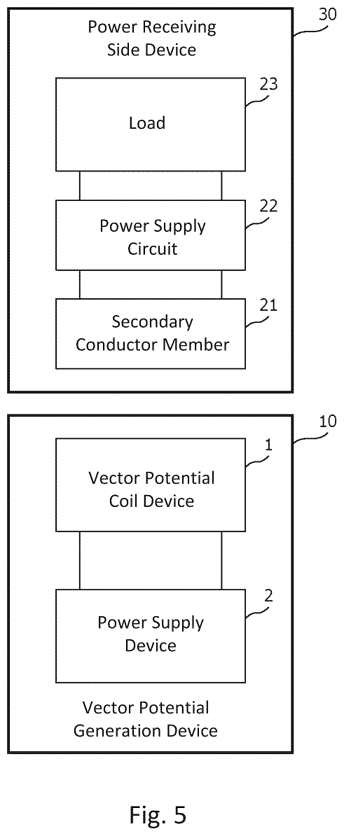

FIG. 5 is a block diagram that shows a configuration of a contactless power supply system according to a second embodiment of the present invention. As shown in FIG. 5, the contactless power supply system according to the second embodiment has the above-mentioned vector potential generation device 10 and a power receiving side device 30. The power receiving side device 30 has a secondary conductor member 21 as an application target of a vector potential, a power supply circuit 22, and a load 23.

The secondary conductor member 21 senses the vector potential being generated due to the AC current by the vector potential generation device 10. Further, as shown in the above-mentioned Patent Document 3, a secondary voltage (AC voltage) is induced due to a temporal change (varying in time) of the vector potential.

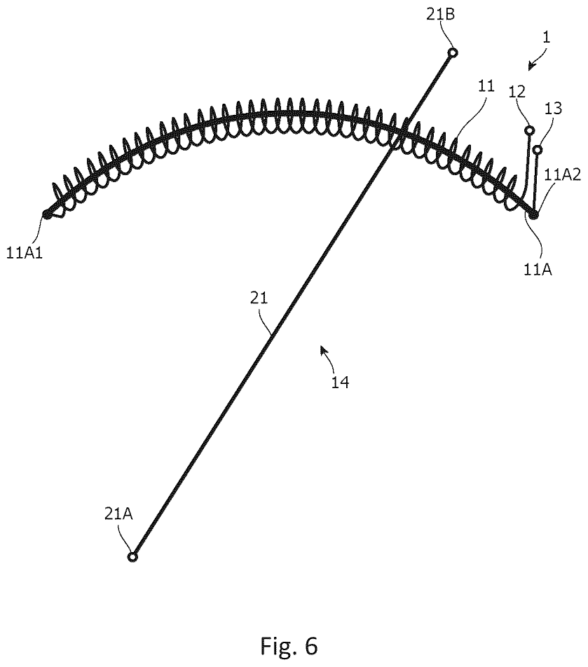

FIG. 6 is a diagram that shows an example of the vector potential coil device 1 and the secondary conductor member 21 in the contactless power supply system shown in FIG. 5.

In this embodiment, as shown in, for instance, FIG. 6, the secondary conductor member 21 is a linear conductive member and is arranged so as to be in parallel to the vector potential being generated by the vector potential generation device 10 (in other words, so as to be in a vertical direction with respect to a plane that includes the coil axis of the VP coil 11).

Further, it is preferred that the secondary conductor member 21 is arranged at the center of the curvature of the coil axis of the VP coil 11.

In addition, the power supply circuit 22 is connected to terminals 21A and 21B of the secondary conductor member 21, and supplies power being obtained by the voltage (that is, the above-mentioned secondary voltage) being generated between the terminals 21A and 21B to the load 23. The power supply circuit 22 has, for instance, a rectification smoothing circuit, converts power being obtained by the secondary voltage into DC power, and supplies the DC power to the load 23.

Next, an operation of the contactless power supply system according to the second embodiment will be explained.

As mentioned in the first embodiment, the vector potential having a relatively great intensity is generated by the vector potential generation device 10 in the secondary conductor member 21 of the power receiving side device 30 that is arranged at the opening 14 or in the periphery of the opening 14. As a result, the secondary voltage is generated in the secondary conductor member 21. The power supply circuit 22 supplies power based on the secondary voltage to the load 23.

As mentioned above, according to the above-mentioned second embodiment, in the power receiving side device 30, the secondary conductor member 21 senses the vector potential being generated by the vector potential generation device 10. The secondary voltage is induced by the vector potential. Further, the power supply circuit 22 supplies power being obtained by the secondary voltage to the load 23.

As a result, power is supplied in the contactless manner from the vector potential generation device 10 to the power receiving side device 30.

### Third Embodiment

A vector potential transformer according to a third embodiment of the present invention has the above-mentioned VP coil 11, the above-mentioned ferromagnetic member 11A, and the above-mentioned secondary conductor member 21. Therefore, power is transmitted from the VP coil 11 (i.e., a primary side) to the secondary conductor member 21 (i.e., a secondary side). The vector potential transformer is a single device that has the VP coil 11, the ferromagnetic member 11A, and the secondary conductor member 21. Thus, a relative positional relationship between the VP coil 11 and the ferromagnetic member 11A, and the secondary conductor member 21 is fixed.

Note that the VP coil 11 that is used in the vector potential transformer according to the third embodiment may be one or plural. Further, any of the VP coils 11 in the other embodiments may be used appropriately.

### Fourth Embodiment

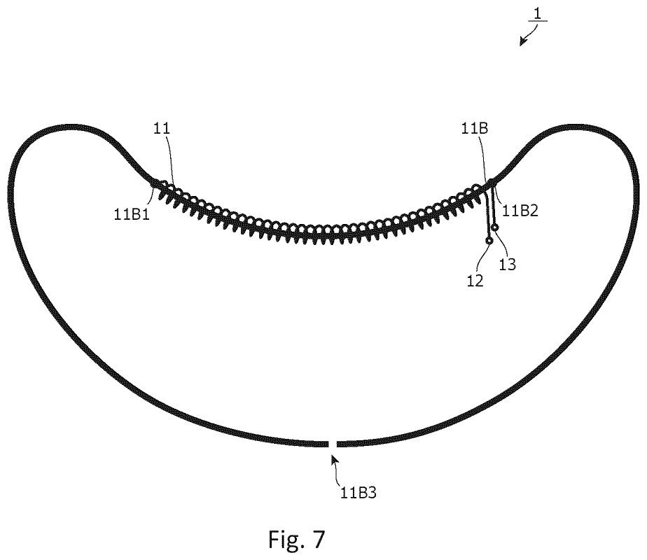

FIG. 7 is a diagram that shows a configuration of a vector potential coil device 1 with respect to a vector potential generation device 10 according to a fourth embodiment of the present invention.

In the fourth embodiment, as shown in, for instance, FIG. 7, a ferromagnetic member 11B is arranged at an inside of a VP coil 11 (i.e., a solenoid coil).

In the same as the ferromagnetic member 11A, the ferromagnetic member 11B is in a shape along the coil axis of the VP coil 11. In addition, the ferromagnetic member 11B extends toward an outside of the VP coil 11 (in the outer side (direction) of the curvature) so as to form a closed magnetic path.

The ferromagnetic member 11B is a member being made of a conductive ferromagnetic material (for instance, a metallic magnetic material such as permalloy), and has a connection point 11B1 on a side of one coil end of the VP coil 11 and a connection point 11B2 on a side of the other coil end of the VP coil 11. Further, the one coil end of the VP coil 11 is electrically connected to the ferromagnetic member 11B at the connection point 11B1.

In addition, the other coil end of the VP coil 11 is electrically connected to a terminal 12. The connection point 11B2 of the ferromagnetic member 11B is electrically connected to a terminal 13 via a lead wire. Further, the power supply device 2 applies a voltage to the terminals 12 and 13 so as to conduct a current through the VP coil 11.

In addition, a gap 11B3 is formed in the ferromagnetic member 11B at the outer side of the curvature of the VP coil 11. Further, the gap 11B3 prevents the current from being conducted through an outside part of the curvature of the VP coil 11 in the ferromagnetic member 11B.

Further, it is preferred that the transition portion between the inner portion and the outer portion of the ferromagnetic member 11B is made into a continuous and smooth curve shape without any steep bend portions, in order to reduce a leakage of a magnetic flux and reduce the influence of the decrease of the magnetic permeability due to bending processing. Moreover, the ferromagnetic member 11B may be formed by connecting a plurality of members.

The vector potential coil device 1 with respect to the vector potential generation device 10 according to the fourth embodiment can be applicable to any of the first to third embodiments.

### Fifth Embodiment

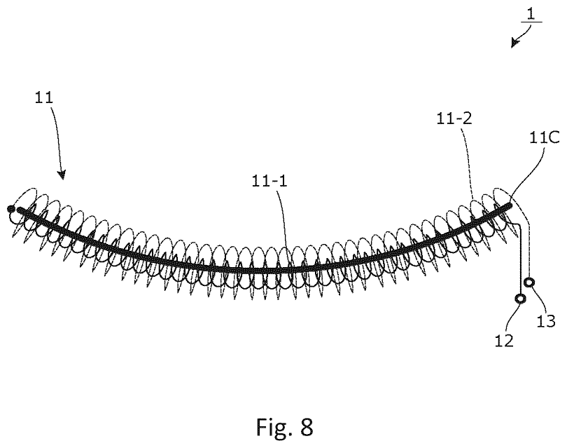

FIG. 8 is a diagram that shows a configuration of a vector potential coil device 1 with respect to a vector potential generation device 10 according to a fifth embodiment of the present invention.

In the fifth embodiment, as shown in, for instance, FIG. 8, the VP coil 11 has an inner solenoid coil 11-1 and an outer solenoid coil 11-2 that respectively extend along the same curved coil axis and in which coil diameters are different from each other.

Further, one coil end of the inner solenoid coil 11-1 and one coil end of the outer solenoid coil 11-2 are electrically connected. Each of the inner solenoid coil 11-1 and the outer solenoid coil 11-2 functions as one VP coil. Therefore, the VP coil 11 according to the fifth embodiment electrically has a configuration in which two VP coils are connected in series and in the same phase.

Furthermore, in the fifth embodiment, as shown in, for instance, FIG. 8, a ferromagnetic member 11C is arranged inside the VP coil 11 (the inner solenoid coil 11-1). The ferromagnetic member 11C is the same as the above-mentioned ferromagnetic member 11A. However, the VP coil 11 and the ferromagnetic member 11C are not electrically connected. Further, the ferromagnetic member 11C may not have to be conductive.

The power supply device 2 applies a voltage to the other end of the inner solenoid coil 11-1 and the other end 11-2 of the outer solenoid coil so that the current is conducted through the VP coil 11. Specifically, the power supply device 2 applies the voltage to the terminal 12 being electrically connected to the other end of the inner solenoid coil 11-1 and the terminal 13 being electrically connected to the other end 11-2 of the outer solenoid coil so that the current is conducted through the VP coil 11.

The vector potential coil device 1 with respect to the vector potential generation device 10 according to the fifth embodiment can be applicable to any of the first to third embodiments.

### Sixth Embodiment

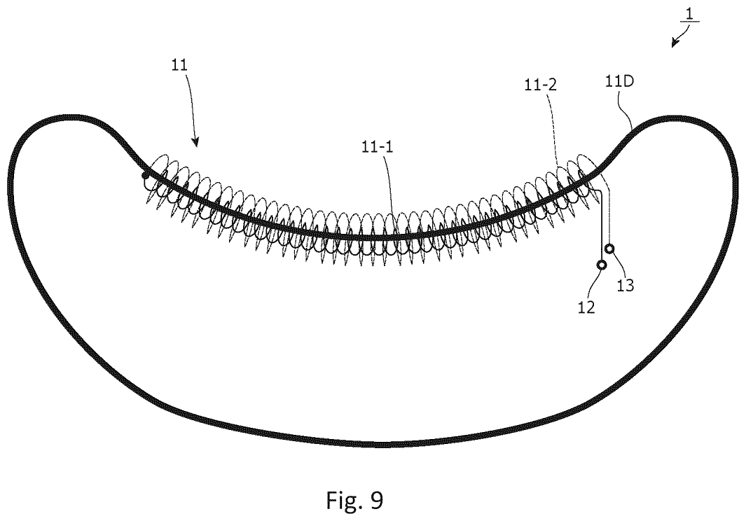

FIG. 9 is a diagram that shows a configuration of a vector potential coil device 1 with respect to a vector potential generation device 10 according to a sixth embodiment of the present invention.

In the sixth embodiment, as shown in, for instance, FIG. 9, the VP coil 11 has the inner solenoid coil 11-1 and the outer solenoid coil 11-2 that are the Same as those in the fifth embodiment. Furthermore, in the sixth embodiment, as shown in, for instance, FIG. 9, a ferromagnetic member 11D is arranged inside the VP coil 11 (the inner solenoid coil 11-1). The ferromagnetic member 11D is the same as the above-mentioned ferromagnetic member 11B. However, the VP coil 11 and the ferromagnetic member 11D are not electrically connected. Further, the ferromagnetic member 11D may not have to be conductive. In addition, a gap is not provided. That is, in the sixth embodiment, since an AC current is conducted through the inner solenoid coil 11-1 and the outer solenoid coil 11-2 but is not conducted through the ferromagnetic member 11D, the ferromagnetic member 11D does not require conductivity or the gap.

Note that the other configurations and operations of the vector potential coil device 1 with respect to the vector potential generation device 10 according to the sixth embodiment are the same as those explained in the fifth embodiment. Therefore, the explanations of them will be omitted.

### Seventh Embodiment

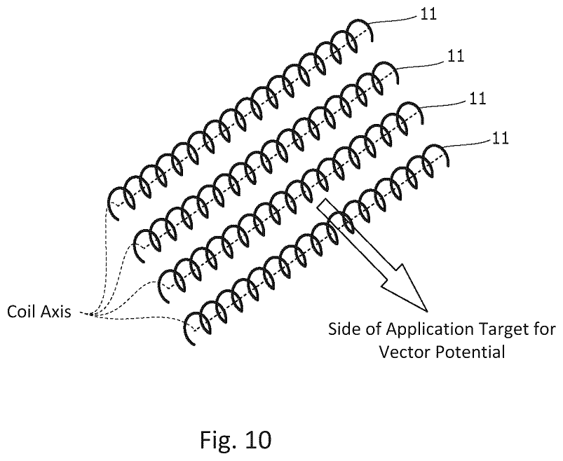

FIG. 10 is a diagram that shows a configuration of a vector potential coil device 1 with respect to a vector potential generation device 10 according to a seventh embodiment of the present invention.

In the seventh embodiment, the vector potential coil device 1 has a plurality 4 VP coils 11. Each VP coil 11 in the seventh embodiment has a linear coil axis. Further, the VP coils 11 are a plurality of solenoid coils extending along the coil axes. The plurality of VP coils 11 are arranged along a linear arrangement direction. In other words, the outer shape of the vector potential coil device 1 is in a substantially flat plate shape. The power supply device 2 conducts a current through the plurality of VP coils 11. Further, the plurality of VP coils 11 may be electrically connected in series or in parallel to one another. Furthermore, a plurality of power supply devices 2 may conduct the current to the plurality of VP coils 11, respectively. In this case, the plurality of power supply devices 2 respectively conduct AC current through the plurality of VP coils 11 so that the AC currents being conducted through the plurality of VP coils 11 are synchronized. Furthermore, the vector potential coil device 1 has a plurality of ferromagnetic members (not shown) that extend along the coil axes of the plurality of VP coils 11 in the same manner as the above-mentioned ferromagnetic members.

As mentioned above, by providing the plurality of VP coils 11, the intensity of the vector potential being applied to the application target becomes greater.

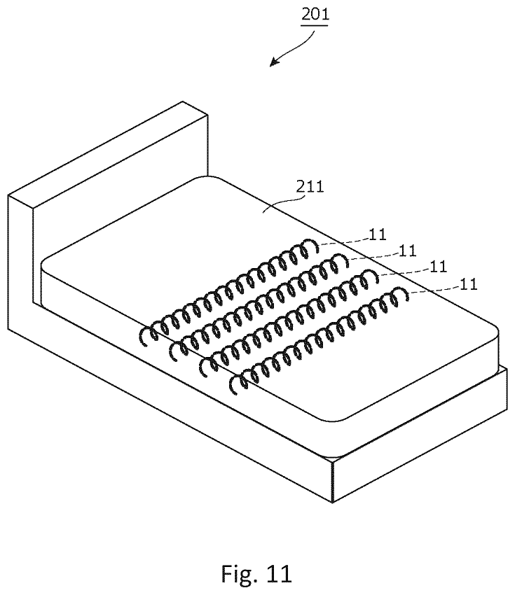

FIG. 11 is a diagram that shows an example of a bed that incorporates the vector potential generation device 10 according to the seventh embodiment. As shown in, for instance, FIG. 11, the above-mentioned plurality of VP coils 11 are incorporated into a mattress 211 of a bed 201. Further, the power supply device 2 is installed in such as a body of the bed 201. As a result, the vector potential is applied to a person lying in the bed 201. Note that in FIG. 11, the VP coils 11 are arranged along the perpendicular direction with respect to the longitudinal direction of the bed 201 (the longitudinal direction of a human body).

However, the VP coils 11 may be arranged along the longitudinal direction of the bed 201 (the longitudinal direction of o the human body). In addition, in FIG. 11, the mattress 211 incorporates the VP coils 11. However, the VP coils 11 may be incorporated in, for instance, the bed body or a pad being placed on the mattress 211.

Note that the other configurations and operations of the vector potential generation device 10 according to the seventh embodiment are the same as those explained in any of the other embodiments. Therefore, the explanations of them will be omitted.

### Eighth Embodiment

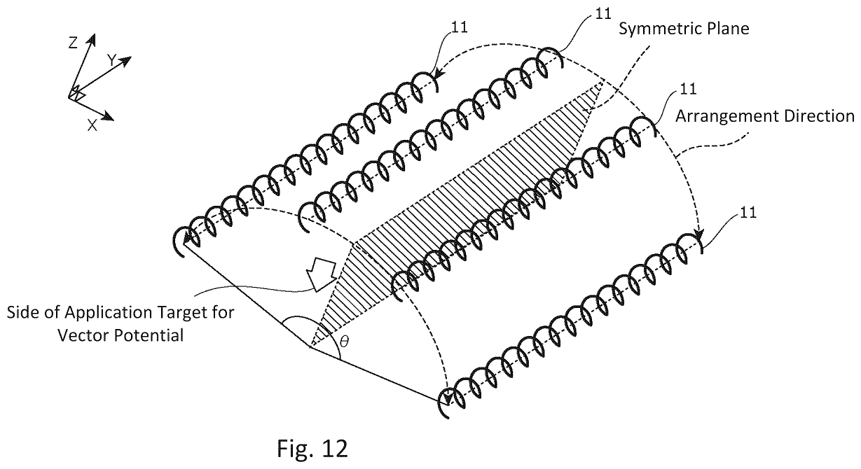

FIG. 12 is a diagram that shows a configuration of a vector potential coil device 1 with respect to a vector potential generation device 10 according to an eighth embodiment of the present invention.

In the eighth embodiment, the vector potential coil device 1 has a plurality of VP coils 11. Each VP coil 11 in the eighth embodiment has a linear coil axis. Further, the VP coils 11 are a plurality of solenoid coils extending along the coil axes. The plurality of VP coils 11 are arranged along a curved (curvature) arrangement direction. The power supply device 2 conducts the current through the plurality of VP coils 11. Further, the plurality of VP coils 11 may be electrically connected in series or in parallel to one another. Furthermore, the vector potential coil device 1 has a plurality of ferromagnetic members (not shown) that extend along the coil axes of the plurality of VP coils 11 in the same manner as the above-mentioned ferromagnetic members. Here, the arrangement direction is a closed curve. Thus, the plurality of VP coils 11 are arranged along the circular-arc-shaped arrangement direction. In particular, the plurality of VP coils 11 are arranged within a range of a predetermined central angle O (here, at an interval of an equal angle) with respect to a circle that includes the circular arc in the arrangement direction. Since the vector potentials of two of the VP coils 11 are canceled out at the intermediate position between the two VP coils 11, for instance, the central angle e is set to be any angle less than 180 degrees.

For instance, a part of the human body such as an arm or a leg is arranged within the space in the inner side (direction) of the arranged plurality of VP coils 11. Thus, the vector potential is applied to that part.

Note that as shown in, for instance, FIG. 12, when the plurality of VP coils 11 having the linear coil axes are arranged plane-symmetrically with respect to a predetermined symmetric plane (a plane perpendicular to the X-axis and parallel to the Z-axis and the Y-axis) along the curved arrangement direction, on an axis that passes through the center of the circle that includes the circular arc in the arrangement direction, and at the same time, is parallel to the coil axes, as a result of a vector synthesis of the vector potentials that are generated by the plurality of VP coils 11, the vector potential is generated in a vertical direction with respect to the symmetric plane (the X-axis direction in FIG. 12). Therefore, for instance, by combining the VP coil 11 having the curved coil axis shown in FIG. 4 with the plurality of VP coils 11 having the linear coil axes and being arranged plane-symmetrically with respect to the predetermined symmetric plane along the curved arrangement direction, it is possible to generate a vector potential in a desired direction within a two-dimensional plane of the X-axis and Y-axis.

Note that the other configurations and operations of the vector potential generation device 10 according to the eighth embodiment are the same as those explained in any of the other embodiments. Therefore, the explanations of them will be omitted.

### Ninth Embodiment

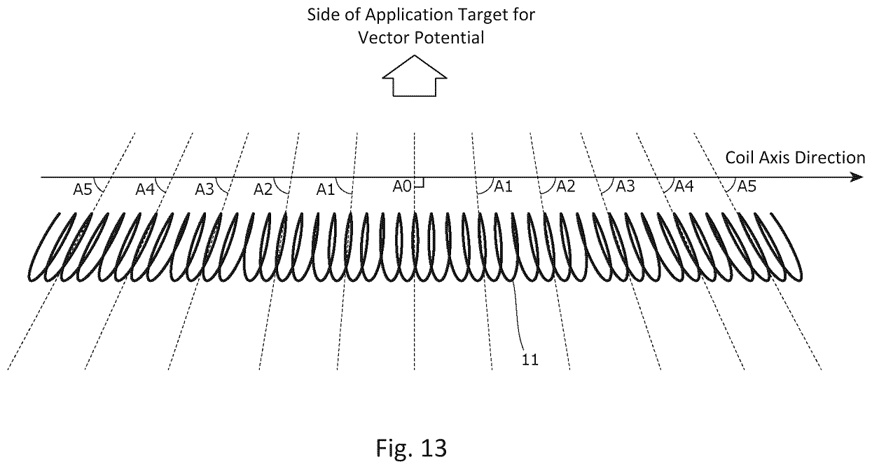

FIG. 13 is a diagram that shows a configuration of a vector potential coil device 1 with respect to a vector potential generation device 10 according to a ninth embodiment of the present invention.

In the ninth embodiment, the vector potential coil device 1 has a plurality of VP coils 11. Each of the plurality of VP coils 11 is wound along (around) a linear coil axis and is wound so that inclination angles in the winding direction gradually change along the direction of the coil axis. Further, the vector potential coil device 1 has a plurality of ferromagnetic members (not shown) that extend along the coil axes of the plurality of VP coils 11 in the same manner as the above-mentioned ferromagnetic members.

In the ninth embodiment, as shown in, for instance, FIG. 13, the VP coil 11 is wound along (around) the linear coil axis, and is wound so that the inclination angles (the angles formed by the coil axis direction and the winding direction) A0-A5 in the winding direction gradually change along the direction of the coil axis. Specifically, the inclination angle at the center of the VP coil 11 is 90 degrees. Further, the inclination angle becomes smaller as it moves away from the center (A0>A1>A2>A3>A4>A5). As a result, the above-mentioned vector potential can be applied with satisfactory intensity in the same manner as the case of the curved VP coil 11.

Note that the other configurations and operations of the vector potential generation device 10 according to the ninth embodiment are the same as those explained in any of the other embodiments. Therefore, the explanations of them will be omitted.

### Tenth Embodiment

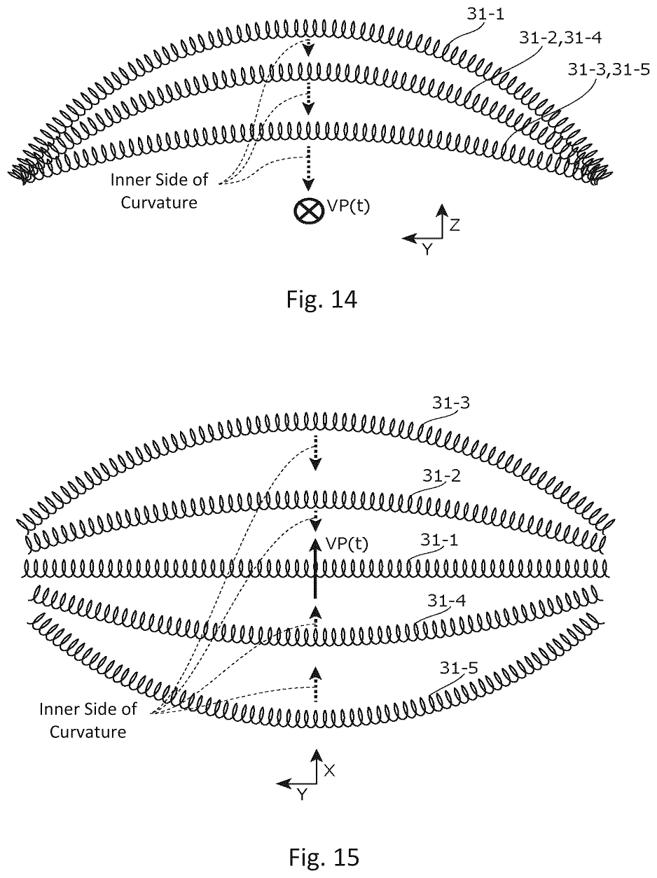

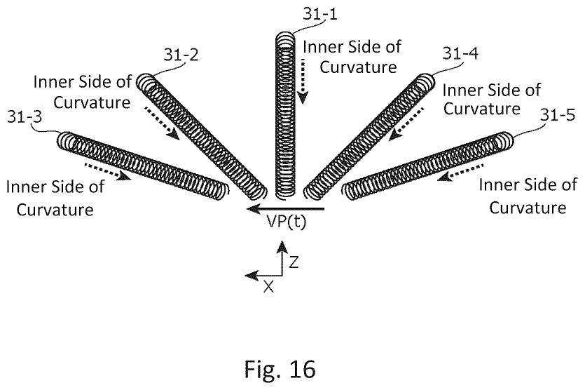

FIG. 14 is a front view that shows an example of a vector potential coil with respect to a vector potential coil device 1 according to a tenth embodiment of the present invention. FIG. 15 is a top view that shows an example of the vector potential coil with respect to the vector potential coil device 1 according to the tenth embodiment of the present invention. FIG. 16 is a side view that shows an example of the vector potential coil with respect to the vector potential coil device 1 according to the tenth embodiment of the present invention.

The vector potential coil device 1 according to the tenth embodiment has a plurality of vector potential coils 31-1-31-5. As shown in, for instance, FIGS. 14-16, the plurality of vector potential coils 31-1-31-5 are respectively wound along (around) a curved coil axis, and are arranged so that the inner sides (directions) of the curvatures of the coil axes (in other words, planes that include the coil axes) cross mutually. For instance, as shown in FIG. 16, the plurality of vector potential coils 31-1-31-5 are arranged so that the planes that include the coil axes of the plurality of vector potential coils 31-1 to 31-5 are parallel to the Y-axis direction, and at the same time, the angle intervals of the inclination angles of these planes with respect to the X-axis direction are substantially the same. In addition, here, the inclination angle of the vector potential coil 31-1 is 90 degrees.

Note that, here, the vector potential coil device 1 has five of the vector potential coils 31-1 31-5. However, the vector potential coil device 1 may have the vector potential coils 31-1-31-M in the same manner as the configuration described above. The number M is either 2-4 coils or 6 or more coils.

For instance, the shape (such as the curvature) and the arrangement of the coil axes are determined so that the coil axes of the plurality of vector potential coils 31-1-31-5 are included in a single partial spherical surface (for instance, a semispherical surface). Further, the application target is arranged at the center of the spherical surface that includes that partial spherical surface (in other words, the center of curvatures of all of the coil axes). Further, the shape (such as the curvature) and the arrangement of the coil axes may be determined so that the coil axes of the plurality of vector potential coils 31-1-31-5 are included in a curved surface (a partial aspherical surface) other than a single partial spherical surface.

Further, the plurality of vector potential coils 31-1-31-5 respectively generate a vector potential according to the AC current in the same manner as the above-mentioned embodiments. The vector potentials by the plurality of vector potential coils 31-1-31-5 are synthesized so that a vector potential VP(t) is obtained. Here, the power supply device 2 conducts the AC current through the plurality of vector potential coils 31-1-31-5 so that the amplitude of the synthesized vector potential VP (t) becomes maximum (for instance, in the same phase mutually).

Note that the other configurations and operations of the vector potential generation device 10 according to the tenth embodiment are the same as those explained in any of the other embodiments. Therefore, the explanations of them will be omitted. In other words, any of the above-mentioned ferromagnetic members may be respectively arranged along the coil axes of the plurality of vector potential coils 31-1-31-5.

As mentioned above, according to the vector potential generation device 10 according to the above-mentioned tenth embodiment, it is possible to concentrate the vector potentials in the inner sides (direction) of the curvatures of the plurality of vector potential coils 31-1-31-5 and apply a high-intensity vector potential to the application target.

### Eleventh Embodiment

In an eleventh embodiment, a vector potential coil device 1 is arranged with respect to a head of a human body in order to treat a brain tumor (such as glioblastoma) of the human body.

For instance, in the eleventh embodiment, the vector potential generation device 10 further has a support body being in a shape to match the shape of the head of the human body. Further, any of the vector potential coil devices 1 mentioned above (for instance, the VP coil, the ferromagnetic member, and the plurality of VP coils) is fixed to the support body. As the support body, for instance, a helmet to be worn on the head or a stand for arranging a VP coil proximate to the head can be used.

Alternatively, the above-mentioned support body may be a device that receives contact with the head, such as a pillow or a headrest of a chair. Further, in this case, the above-mentioned vector potential coil device 1 is incorporated into such the support body.

With such the support body, the vector potential coil device 1 is arranged proximate to a position in which a vector potential is generated in the human brain. In other words, since an AC current is conducted through the vector potential coil device 1, an alternating vector potential is generated in the brain inside the head. As a result, an AC electric field or an AC current is applied to the brain. For instance, as disclosed in International Patent Publication Number WO2017/072706, brain tumors are treated by applying an AC electric field. The conditions (such as a frequency) being required for treating the brain tumors are set by the power supply device 2. Further, the AC electric field under such conditions is applied non-invasively to the brain by the vector potential coil device 1. For instance, as disclosed in International Patent Publication Number WO2017/072706, in order to apply the AC electric field, electrode pads are usually attached to the skin of the head after shaving hair. However, according to the eleventh embodiment, shaving hair is not required, and at the same time, it is not required to attach adhesive electrode pads to the skin of the head. Thus, the burden (physical burden and mental burden) on the patient during the treatment by applying the AC electric field is reduced.

In the conventional method in which a current is supplied by contacting electrode pads with the scalp, the contact resistance between the pads and the scalp and the electric resistance of the skull are high. On the other hand, brain tissue has a relatively low resistance in comparison with the above. Therefore, it was essentially difficult to control the flow of the current to the desired area even when the position of the electrode pads was changed. On the other hand, there is also another conventional method in which a pulse magnetic field is applied from the outside in the contactless manner and in the non-invasive manner. However, what is induced in the brain is an eddy current. According to Faraday's electromagnetic induction and Lenz's law, this eddy current flows in a direction in which the change in the magnetic field is canceled out. Therefore, because the original pulse magnetic field was canceled out by the eddy current flowing near the epidermis of the cerebrum, it was impossible to induce current in the deep part inside the brain. In addition, the cerebrum has a wrinkled structure in which the surface is folded. Thus, even when the wrinkles are adjacent, the valley between wrinkles is deep. With respect to the electricity, the electric resistance between the wrinkles is high. Therefore, it is difficult to flow current to the desired locations by using a pulse magnetic field. Furthermore, since the magnetic flux of the pulse magnetic field is applied from a direction perpendicular to the scalp, the eddy currents can be flown only in a plane being parallel to the scalp. The two conventional methods have the above-mentioned problems. However, since the device according to the above embodiments generates a vector potential, it is possible to apply an electric field to even deep parts of the brain, for instance, the cerebellum, corpus callosum, and hypothalamus, without being obstructed by the skull.

Note that the other configurations and operations of the vector potential generation device 10 according to the eleventh embodiment are the same as those explained in any of the other embodiments. Therefore, the explanations of them will be omitted.

Note that various changes and modifications to the embodiments described above will be apparent to one having ordinally skill in the art. Such the changes and modifications may be made without departing from the spirit and scope of the subject matter and without diminishing the intended advantages. That is, it is intended that such the changes and modifications are included within the scope of the claims.

For instance, in the above-mentioned embodiments, there may be an object having a magnetic shielding property between the VP coil 11 and the application target of the vector potential (for instance, the human foot 101 or the secondary conductor member 21). Even in this case, the vector potential penetrates the object having the magnetic shielding property. Thus, even when there is the object having the magnetic shielding property, the vector potential is applied to the application target of the vector potential. Further, a voltage based on the vector potential is induced through the secondary conductor member 21.

Further, in the above-mentioned embodiments, the ferromagnetic member that is arranged along the coil axis of the VP coil 11 may be omitted as necessary.

Furthermore, in the above-mentioned second embodiment, the power is transmitted from the vector potential generation device 10 to the secondary conductor member 21. However, in addition to the above, during a period of time in which the power is not transmitted from the vector potential generation device 10 to the secondary conductor member 21, an AC current (for instance, a regenerative current) may be conducted through the secondary conductor member 21 so that power is transmitted from the secondary conductor member 21 to the vector potential generation device 10.

In addition, in the above-mentioned fifth and sixth embodiments, the VP coil 11 has the two-layer structure, which are the inner solenoid coil 11-1 and the outer solenoid coil 11-2, in the radial direction. However, the number of layers may be four or more as long as the number of layers is an even number. In this case, either end of the ends of the solenoid coil 11-i is connected to the solenoid coil 11-(i+1) in the next layer so that the solenoid coils 11-i in all layers are electrically connected in series.

### INDUSTRIAL APPLICABILITY

The present invention can be applicable to, for instance, the generation of a vector potential by using a VP coil.

1. A vector potential generation device comprising:
a vector potential coil that is a solenoid coil extending along a curved coil axis;
a ferromagnetic member extending along the coil axis within the solenoid coil; and
a power supply device that conducts a current through the vector potential coil,
wherein the vector potential coil and the ferromagnetic member have an opening in a circumferential direction.

2. The vector potential generation device according to claim 1, further comprising:
a plurality of vector potential coils including the vector potential coil and being a solenoid coil extending along a curved coil axis; and
a plurality of ferromagnetic members respectively extending along the coil axis within the solenoid coil of the plurality of vector potential coils, wherein the power supply device conducts a current through the plurality of vector potential coils.

3. The vector potential generation device according to claim 1,
wherein the coil axis is in a circular arc shape, and
an angle from one end to the other end of the vector potential coil when viewed from a center of a circle including the coil axis is less than 360 degrees.

4. The vector potential generation device according to claim 1,
wherein the ferromagnetic member is conductive,
one end of the vector potential coil and one end of the ferromagnetic member are electrically connected to each other, and
the power supply device conducts the current through the vector potential coil by applying a voltage to the other end of the vector potential coil and the other end of the ferromagnetic member.

5. The vector potential generation device according to claim 1,
wherein the ferromagnetic member extends toward an outside of the vector potential coil to form a closed magnetic path.

6. The vector potential generation device according to claim 1,
wherein the vector potential coil has an inner solenoid coil and an outer solenoid coil that respectively extend along the same curved coil axis,
one end of the inner solenoid coil and one end of the outer solenoid coil are electrically connected, and
the power supply device conducts the current through the vector potential coil by applying a voltage to the other end of the inner solenoid coil and the other end of the outer solenoid coil.

7. The vector potential generation device according to claim 1, further comprising:
a support body supporting the vector potential coil and the ferromagnetic member,
wherein the support body arranges the vector potential coil and the ferromagnetic member at a position at which a vector potential is generated in a brain of a human body without attaching the support body to a head of the human body.

8. A vector potential coil arrangement method comprising:
arranging a vector potential coil and a ferromagnetic member at a position at which a vector potential is generated in a brain of a human body via a support body without attaching the support body to a head of the human body, the support body supporting the vector potential coil, which is a solenoid coil extending along a curved coil axis, and the ferromagnetic member extending along the coil axis within the solenoid coil.

9. A vector potential generation device comprising:
a plurality of vector potential coils, which are a plurality of solenoid coils extending along their respective coil axes; and
a power supply device that conducts a current to the plurality of vector potential coils,
wherein the plurality of vector potential coils are then arranged along a linear arrangement direction or a curved arrangement direction.

10. The vector potential generation device according to claim 9, further comprising:
a plurality of ferromagnetic members extending along the coil axes within the plurality of solenoid coils, respectively.

11. The vector potential generation device according to claim 9,
wherein the plurality of vector potential coils are respectively wound along a linear coil axis and are wound so that inclination angles in a winding direction gradually change along a direction of the coil axis.

12. The vector potential generation device according to claim 9,
wherein the plurality of vector potential coils are arranged along a circular-arc-shaped arrangement direction and within a range of a predetermined central angle with respect to a circle that includes a circular arc in the arrangement direction, and
the predetermined central angle is any angle less than 180 degrees.

13. The vector potential generation device according to claim 9,
wherein the plurality of vector potential coils are respectively wound along a curved coil axis and are arranged so that inner side directions of curvatures of the coil axes cross one another.

14. The vector potential generation device according to claim 9, further comprising:
a support body supporting the plurality of vector potential coils,
wherein the support body arranges the plurality of vector potential coils at a position at which a vector potential is generated in a brain of a human body without attaching the support body to a head of the human body.

15. A vector potential coil arrangement method comprising:
arranging a plurality of vector potential coils at a position at which a vector potential is generated in a brain of a human body via a support body without attaching the support body to a head of the human body, the support body supporting the plurality of vector potential coils, which are a plurality of solenoid coils extending along respective coil axes.

16. A contactless power supply system comprising:
the vector potential generation device according to claim 1; and
a power receiving side device,
wherein the power receiving side device senses a vector potential generated by the vector potential generation device and has a secondary conductor member, in which a secondary voltage is induced by the vector potential, and a power supply circuit that supplies power obtained from the secondary voltage to a load.

17. A vector potential transformer comprising:
a vector potential coil that is a solenoid coil extending along a curved coil axis;
a ferromagnetic member extending along the coil axis within the solenoid coil; and
a secondary conductor member that senses a vector potential generated by the vector potential coil and in which a secondary voltage is induced by the vector potential,
wherein the vector potential coil and the ferromagnetic member have an opening in a circumferential direction.

18. A vector potential transformer comprising:
a plurality of vector potential coils that are a plurality of solenoid coils extending along coil axes, respectively; and
a secondary conductor member that senses a vector potential generated by the plurality of vector potential coils and in which a secondary voltage is induced by the vector potential,
wherein the plurality of vector potential coils are arranged along a linear arrangement direction or a curved arrangement direction.

19. A contactless power supply system comprising:
the vector potential generation device according to claim 9; and
a power receiving side device,
wherein the power receiving side device senses a vector potential generated by the vector potential generation device and has a secondary conductor member, in which a secondary voltage is induced by the vector potential, and a power supply circuit that supplies power obtained from the secondary voltage to a load.
